(19) United States
(12) Patent Application Publication
(10) Pub. No.: US 2016/0144194 A1
(43) Pub. Date: May 26, 2016

(54) DETERMINING EFFECTIVE ELECTRODES FOR ELECTRICAL STIMULATION

(71) Applicant: MEDTRONIC BAKKEN RESEARCH CENTER B.V., MAASTRICHT (NL)

(72) Inventors: Jonas J. Roothans, EINDHOVEN (NL); Robin Brouns, EINDHOVEN (NL); Rutger Nijlunsing, VELDHOVEN (NL); Mattias Bengt Johan Astrom, VASTERAS (SE)

(21) Appl. No.: 14/943,971

(22) Filed: Nov. 17, 2015

Related U.S. Application Data
(60) Provisional application No. 62/084,382, filed on Nov. 25, 2014.

(51) Int. Cl. A61N 1/372; A61N 1/05

(52) U.S. Cl. CPC A61N 1/37247; A61N 1/0534

(57) ABSTRACT

In one example, a computing device includes a memory to store data representative of an implant location of a lead in a patient, wherein the lead comprises a plurality of electrodes, and one or more processors configured to determine, based on the implant location, probabilities of effectiveness of electrical stimulation delivered via each of the electrodes, and present a visual representation of the determined probabilities. For instance, the representation of the determined probabilities may be displayed in conjunction with visual representations of the lead and the electrodes. The electrodes may comprise sectors of rings along the lead. In this manner, a clinician or other user of the programmer device may quickly determine electrodes that will most probabilistically deliver effective therapy via electrical stimulation.

This application claims the benefit of U.S. Provisional Application No. 62/084,382, filed Nov. 25, 2014, which is hereby incorporated by reference in its entirety.

### TECHNICAL FIELD

This disclosure relates to implantable medical devices, and more particularly, to implantable leads.

### BACKGROUND

Implantable neurostimulation devices have been used to treat acute or chronic neurological conditions. Deep brain stimulation (DBS), the mild electrical stimulation of sub-cortical structures, belongs to this category of implantable devices, and has been shown to be therapeutically effective for Parkinson's disease, Dystonia, and Tremor. New applications of DBS in the domain of psychiatric disorders (obsessive compulsive disorder, depression) are being researched and show promising results. In existing systems, the probes are connected to an implantable current pulse generator.

### SUMMARY

In general, this disclosure describes techniques for determining effective electrodes for electrical stimulation therapy applied to a patient, e.g., via an implantable medical device (IMD). The IMD may control one or more leads, each having one or more electrodes. The electrodes may correspond to sectors of rings along the lead or to leads having both sectors of rings and full ring electrodes. A programmer device may determine where the lead is implanted in a patient's brain and warp scans of the patient's brain to fit an atlas of the brain (or an atlas of a region of the brain). The programmer device may also determine a desired stimulation zone of the brain. The programmer device may then determine probabilities of therapeutic effectiveness for the electrodes (e.g., alone or in combination) when delivering electrical stimulation, e.g., based on the implant location of the lead, the positions of the electrodes and the targeted stimulation zone. The programmer device may further present graphical representations of the determined probabilities, e.g., in conjunction with representations of the lead and electrodes.

In one example, a method includes determining an implant location of a lead in a patient, wherein the lead comprises a plurality of electrodes, determining, based on the implant location, probabilities of effectiveness of electrical stimulation delivered via each of the electrodes, and presenting a visual representation of the determined probabilities.

In another example, a programmer device includes a memory to store data representative of an implant location of a lead in a patient, wherein the lead comprises a plurality of electrodes, and one or more processors configured to determine, based on the implant location, probabilities of effectiveness of electrical stimulation delivered via each of the electrodes, and present a visual representation of the determined probabilities.

In another example, a computer-readable storage medium has stored thereon instructions that, when executed, cause a processor of a programmer device to determine an implant location of a lead in a patient, wherein the lead comprises a plurality of electrodes, determine, based on the implant location, probabilities of effectiveness of electrical stimulation delivered via each of the electrodes, and present a visual representation of the determined probabilities.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a schematic drawing of a neurostimulation system for deep brain stimulation (DBS).

FIGS. 2A-2C are further schematic drawings of example probes of a neurostimulation system for deep brain stimulation (DBS) and their components.

FIG. 3 is a schematic drawing of a probe system according to the techniques of this disclosure.

FIG. 4 is a schematic drawing of a display of a medical system as a part of a neural application system according to the techniques of this disclosure, with which a method according to these techniques can be performed.

FIG. 5 is a further schematic drawing of a display of a medical system as a part of a neural application system according to the techniques of this disclosure, with which a method according to these techniques can be performed.

FIG. 6 is a block diagram illustrating components of an example implantable medical device (IMD).

FIG. 7 is a functional block diagram illustrating components of an example medical device programmer device.

FIG. 8 is a flowchart illustrating an example method that may be used when performing the techniques of this disclosure.

### DETAILED DESCRIPTION

Currently, systems are being developed to include more, smaller electrodes in a technology based on thin film manufacturing. These systems include a lead having electrodes made from a thin film based on thin film technology. The thin film leads are fixed on a core material to form the lead, which may be used in one or more probes. These probes may have multiple electrode areas and may enhance precision to address an appropriate target in a patient's brain, or other implant site, and relax specifications regarding positioning of the probes relative to brain structures targeted for stimulation. Meanwhile, undesired side effects due to undesired stimulation of neighboring areas can be reduced.

The generated stimulation field may be applied to selected brain regions in order to directly change brain activity in a controlled manner. The location and distribution of the stimulation field influences the therapeutic effect.

During implantation of the probe, the physician or surgeon recognizes by means of immediate patient feedback to the stimulation, such as reduction or increase of local tremor, whether the location of the probe results in a desired effect or undesired side-effect. As such, existing methods for planning the implantation of the neuromodulation probe or adapting electrical stimulation parameters are primarily based upon a trial and error approach which, however, disregards delayed feedback or clinical outcome. Furthermore, according to the current practice with existing stimulation leads having only a few (n) electrodes (e.g., number n of electrodes<10), clinicians perform a full sweep of all possible combinations during programming sessions with their patient in order to guarantee they find the best outcome. Iterating through all combinations has typically been shown to last around two hours with these leads. With the introduction of leads with a plurality of electrodes (e.g., number n of electrodes>10, especially n>20), ten times many combinations will become available, with a tuning session potentially lasting 20 hours.

The techniques of this disclosure are generally directed to planning and/or tuning a neural stimulation application. Planning and tuning may include selecting electrodes or combinations of electrodes to be used for delivery of electrical stimulation, along with electrode polarities, e.g., anodic or cathodic, and selecting parameters of stimulation delivered via the electrodes, such as voltage or current amplitude, pulse rate (i.e., frequency), and pulse width. Through application of these techniques, the process of adapting the stimulation parameters and the adjustment process of the stimulation field, e.g., to promote therapeutic efficacy, may be simplified and may be less time consuming for the clinician and patient.

An example of a medical system in accordance with the techniques of this disclosure may include:

a database means for storing a plurality of data sets, each data set comprising:
metadata, e.g., patient data, especially relating to gender data, age data, brain activity data—especially local field recordings data and microelectrode recordings data—, tissue data—especially fiber structures data—, demographics data and/or anatomy data of a patient;
positioning data of at least one implanted neurostimulation lead; and
spatial data relating to the possible distribution of at least one stimulation field of the at least one neurostimulation lead;

calculation means for calculating possible stimulation field setting possibilities based on the metadata, the positioning data and the spatial data; and

preselecting means for preselecting the possible stimulation field setting possibilities based on the metadata, the positioning data and the spatial data.

The medical system may be a medical device, for planning and/or tuning a neurostimulation therapy.

The medical system may be configured to rank stimulation field setting possibilities according to the predicted outcome based on the metadata, the positioning data and the spatial data and to weed out stimulation field settings that are ranked lower and deemed to provide no effect or less effect than the higher ranked stimulation field setting possibilities. Thus, the number of possible stimulation settings is reduced to preselected stimulation field setting possibilities which are deemed to be more effective than others. The stimulation field settings may, for example, define stimulation field generated by stimulation current delivered via a plurality of electrodes, such that the amplitude at each electrode contributes to the field shape, size, and the like. The electrodes that work together may have different amplitudes, but may have the same frequency and pulse width. In this way, an anterior and lateral electrode, for example, could produce a combined field with a certain shape (e.g., 30% of maximum stimulation laterally and 50% of maximum stimulation anteriorly, such that a field lobe that slants toward anterior is produced). Consequently, the physician may be given some guidance as to which stimulation field setting possibilities should be tested and also the number of stimulation field setting possibilities that the physician should test is reduced. By this, the process of adapting the stimulation parameters and the adjustment process of the stimulation field is simplified and less time consuming as a lower number of possibilities must be tested than would otherwise be the case if all possible combinations offered by the plurality of stimulation settings must be tested.

The metadata may be at least partially functional brain atlas data and/or other functional data such as data related to functional Magnetic Resonance Imaging (fMRI) and/or fiber tracking such as Diffusion Tensor Imaging (DTI) fiber tracking. This metadata may be used to determine stimulation targets, as discussed below.

The database means may be a database, stored in a data storage device, that contains statistical data of patients who have been treated with a neuromodulation probe, and in particular with a neuromodulation probe of a deep brain stimulation system. This data may be used to generate a functional brain atlas (FBA). For example, the FBA uses a certain amount of data sets, each data set representing a specific patient of a group of patients which have been treated with the neuromodulation probe. Each data set may comprise metadata of a specific patient and/or patient group and spatial data about the distribution of a stimulation field which has been generated during therapy of said patient and/or patient group. The metadata may also include information about the clinical outcome of each patient.

Each data set may also include spatial data related to the stimulation field applied to the specific patient which provided the metadata of said data set. The distribution of the stimulation field may be determined by setting up a coordinate system, preferably a Cartesian coordinate system, aligned to the probe and defining a probe grid. Each point of the probe grid which includes spatial data related to the stimulation field defines the volume of the stimulation field. Spatial data may be assigned only to probe grid points which are covered by the stimulation field. Hence, only spatial data which has been assigned to probe grid points, i.e., which represent probe grid points covered by the stimulation field, may be combined with metadata in a data set.

The calculation means may be one or more processors that calculate, in a first step based on the positioning data and the spatial data, all possible stimulations field setting possibilities that can be generated with the electrodes of the lead. This calculation may for example take into account that the polarity of electrodes may be changed, e.g., that electrodes may be changed from anodic to cathodic electrodes or vice versa, or that electrodes are switched on or off or the like, to deliver or not deliver electrical stimulation current. Furthermore, the calculation may take into account that the stimulation current provided by one of the plurality of electrodes is higher or lower than e.g., the stimulation current provided by another electrode of the plurality of electrodes, which may affect the size and/or shape of a stimulation field produced by the combined stimulation currents sourced or sunk by the electrodes. In particular, the calculations means may calculate all possible shapes and forms of stimulation fields that may be generated by the electrodes of the stimulation lead, where such electrodes may be arranged at various axial and circumferential positions around the lead.

In connection with the calculation means, the preselecting means may be provided by one or more processors configured to preselect the possible stimulation field setting possibilities based on the metadata on the one hand and the positioning data and the spatial data on the other hand. Especially, the preselecting means may use the data provided by the calculation means, which are based on the positioning data and the spatial data and may for example consists of all possible shapes and forms of stimulation fields that may be generated by the electrodes of the stimulation lead as specified above.

These possible shapes and forms of stimulation fields may be checked against the metadata to filter out some or all stimulation settings that do not match or do not sufficiently match to functional areas of tissue, e.g., brain tissue, in order to restrict the number of possible stimulation field settings for the physician or user. For example, some stimulation settings that are not likely to direct stimulation to functional areas of tissue, and thereby not likely to elicit a therapeutically efficacious response, may be eliminated from consideration when selecting stimulation settings. Again, the stimulation settings may comprise electrode combinations, polarities, voltage or current amplitudes, pulse rates and pulse widths for electrical stimulation pulses applied via selected electrodes of the probe.

By filtering the data sets, a filtered subset of the data sets is generated which includes data sets of patients matching the entered filter criterion or criteria. Thus, the techniques of this disclosure may allow a user, e.g., a physician, such as a neurologist, to identify possible stimulation field settings of interest.

Alternatively, the calculation means may calculate a sparse subset of all shapes and forms of stimulation fields that may be generated by the electrodes of the stimulation lead, as a first approximation, and refine further subsets of shapes as subsequent approximations after initial preselections have been made, e.g., to accelerate calculations and converge towards optimal preselections by successive approximations, or by any other convergence acceleration algorithms.

The medical system advantageously allows guidance during surgical planning of a neuromodulation therapy, in particular of probe implantation, intra-operative test stimulation, and postoperative stimulation tuning.

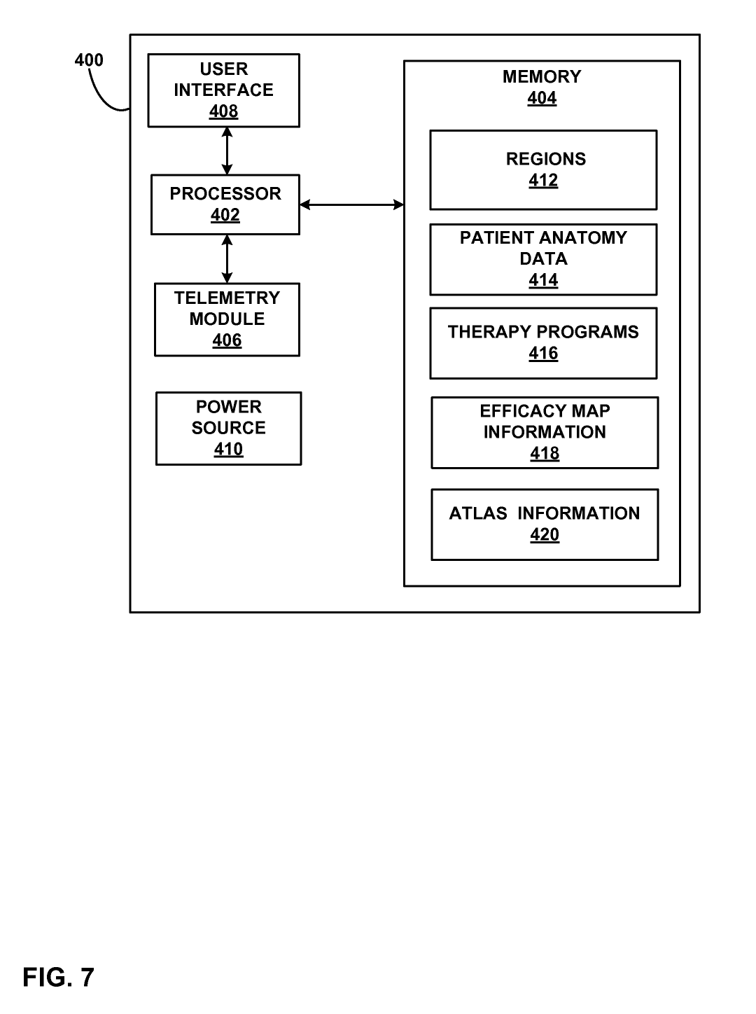

The preselecting means (e.g., one or more processors, such as processor 402 of FIG. 7 as discussed below) may be configured to provide a starting point for the first stimulation chosen from the number of preselected possible stimulation field setting possibilities by preselecting the possible stimulation field possibilities based on the metadata, the positioning data and the spatial data. The preselection of a promising starting point for stimulation field settings, which may be selected by the preselecting means, may, e.g., decrease significantly the duration and complexity of the tuning procedure. For example, the starting point for the first stimulation may be a position within a target region of tissue to be stimulated. This position may be, e.g., chosen such that it is located substantially in a central region of the target region.

The preselecting means may be configured to provide an estimate for the probability of acceptable stimulation results, e.g., maximum therapeutic effect or minimal side effects or the like, based on the metadata, the positioning data and the spatial data. By providing an estimate for the probability of acceptable stimulation results, the user of the system—e.g., the physician or clinician—may influence his optimization and his search for better stimulation settings such as, for example, electrode combinations, polarities, voltage or current amplitudes, pulse rates and pulse widths for electrical stimulation pulses applied via selected electrodes of the probe.

Furthermore, the preselecting means may be configured to provide a limitation of the amount of stimulation options by preselecting possible stimulation field possibilities based on the metadata, the positioning data and the spatial data. A limitation of the amount of stimulation options further assists the user of the system, e.g., the physician, to find optimal stimulation setting more quickly. The limitation may be conducted by the preselecting means by filtering the possible stimulation settings, for example, wherein stimulation settings are proposed when their matching possibility to a target region of tissue to be stimulated is considered to be above a predetermined threshold value, which describes the likelihood that the stimulation settings will match to the target region of tissue on the basis of the metadata, the positioning data and the spatial data. This threshold value and likelihood may be calculated by the calculation means.

The medical system may comprise output means, such as a display, for directionally displaying the areas with increased likelihood for best outcome based on the metadata, the positioning data and the spatial data. By directionally displaying the areas with increased likelihood for best outcome the user, e.g., the physician, is given some guidance on which direction will very likely lead to a desired therapeutic result, i.e., optimal stimulation settings. As examples, the desired therapeutic result may be excitation of brain activity or signals, inhibition of brain activity or signals, or other modifications of brain activity or signals. Accordingly, the stimulation settings may be selected to achieve these types of results. By this, the advantage is achieved that options and directions with less promising outcome will be neglected by the user and that the user may directly focus on the most or more promising directions for adaptation of the possible stimulation settings.

Additionally, the output means may be configured to display sectors around the lead for displaying the areas with increased likelihood for best outcome based on the preselection of possible stimulation field possibilities and based on the metadata, the positioning data and the spatial data. This may help the user to work with the medical system more intuitively. The displaying of sectors is in particular helpful to provide fast and intuitively understandable information about the areas with increased likelihood for best outcome.

Also, the medical system may comprise stimulation means (e.g., electrodes of a lead) for simulating the effect of at least one possible stimulation field setting possibility. By this, the setup procedure may be further enhanced and simplified. The simulation capability offers the user a mode of use, where it is not necessary that, e.g., a patient is connected to the medical system. Thus, this mode may be used to train users or to prepare surgical procedures and setup procedures in order to save time.

Furthermore, an example of the techniques of this disclosure may include a neural application system, especially a deep brain stimulation (DBS) system, comprising an implantable deep brain stimulation lead, which also may be referred to as a probe, and a medical system as specified above and according to the present disclosure, wherein the lead and the medical system are wired and/or wirelessly connected.

The neural application system may comprise all structural and functional features and also all advantages as specified above in connection with the medical system for planning and/or tuning a neural application according to the techniques of this disclosure.

The lead may comprise a plurality of electrodes. A relatively large number of electrodes may provide an advantage in that the shape and form of the stimulation field may be adjusted and formed with higher accuracy than would be possible with a lead with a relatively small number, such as only a single electrode. A relatively large number of electrodes on the lead may be, for example, given at a number of more than 10 electrodes, especially more than 20 electrodes, and up to approximately 40 electrodes, or even more electrodes. Hence, the lead may include more than 10 electrodes, more preferably 20 to 40 electrodes, even more preferably 30 to 40 electrodes, and still more preferably approximately 40 electrodes.

The electrodes may form a complex electrode array. This is helpful to create a stimulation field that is adapted to and conforms to the target region.

A complex electrode array generally refers to an arrangement of electrodes at multiple non-planar or non-coaxial positions, in contrast to simple electrode array geometries in which the electrodes share a common plane or common axis.

An example of a simple electrode array geometry is an array of electrodes distributed at different axial positions along the length of the lead, where each segmented electrode extends about a circumference of the lead at the respective axial position.

An example of a complex electrode array geometry, in accordance with this disclosure, is an array of electrodes positioned at different axial positions along the length of the lead, as well as (alternatively or additionally) at different angular positions about the circumference of the lead. For example, a complex electrode array geometry may include multiple rings of electrodes at different axial positions, where a given ring includes multiple (e.g., two, three, four, six, eight or more) separate electrodes at substantially the same axial position but at different angular positions around the circumference of the lead, e.g., possibly appearing as a segmented electrode divided into individual electrode segments. In this case, each separate electrode segment may be selected separately to deliver electrical stimulation separately from the other electrodes. In these examples, the individual electrodes in the complex electrode geometry are positioned at different angular positions and face outward in different directions, thereby permitting stimulation to be selectively emitted in different directions by selection of individual electrodes for delivery of the stimulation.

In general, this disclosure refers to a set of electrodes at a common distance from a distal tip of an electrode at various axial positions as a segmented electrode or a ring of segmented electrodes. It should be understood, however, that these terms are not intended to imply that the electrodes of such a ring are equidistant from each other about the circumference of the lead. Likewise, when two or more rings of segmented electrodes are provided, each of the rings need not include the same number of electrodes, nor are the electrodes of one ring necessarily commonly positioned with electrodes of another ring (i.e., at common axial positions). Thus, the terms “segmented electrode” and “ring of segmented electrodes” should generally be understood to refer to a set of one or more electrodes at a common distance from a distal tip of a lead, without implying anything more regarding the positioning of the electrodes about the circumference of the lead.

In some examples, the electrodes of a lead according to this disclosure may include one or more full ring electrodes that extend all the way around the circumference of the lead in combination with one or more segmented electrodes of the types discussed above. An example would be a “1-3-3-1” lead. This type of lead has a distal ring or distal tip electrode, and two rows each of three segmented electrodes. The lead may also include a more proximal ring electrode proximal to the two rows of three segmented electrodes. Such a lead is described in U.S. Pat. No. 7,668,601 assigned to the assignee of the current application and incorporated herein by reference. Another example is a “1-N-N-1” lead, which includes a ring electrode at a proximal end of the lead, a ring electrode at a distal end of the lead, and two rings of segmented electrodes, each of the rings of segmented electrodes including N electrodes (where N is an integer number). In still other examples, the complex electrode array may comprise segmented electrodes formed using thin film techniques and the array may comprise any number of electrodes, such as forty or more electrodes. Such electrodes need not take the shape of a partial ring, but may have a different shape.

Moreover, another example of the techniques of this disclosure includes a method of planning and/or tuning a neural application, such as a neurostimulation therapy like a Deep Brain Stimulation Therapy. In one example, a method includes:

storing a plurality of data sets in a database means, each data set comprising:
metadata, e.g., patient data, especially relating to gender data, age data, local field recordings data, microelectrode recordings data, tissue fiber structures data, demographics data and/or anatomy data of a patient, especially wherein the metadata are at least partially functional brain atlas data;
positioning data of an implanted neurostimulation lead; and
spatial data relating to the possible distribution of at least one stimulation field of the neurostimulation lead;

calculating possible stimulation field setting possibilities; and

preselecting the possible stimulation field setting possibilities based on the metadata, the positioning data and the spatial data.

The method may comprise all features and also all advantages as specified above in connection with the medical system for planning and/or tuning a neural application according to the techniques of this disclosure. In particular, the method may be conducted with the medical system for planning and/or tuning a neural application according to the techniques of this disclosure.

The method may further comprise at least one of the following steps:

providing a starting point for the first stimulation chosen from the number of preselected possible stimulation field setting possibilities by preselecting the possible stimulation field possibilities based on the metadata, the positioning data and the spatial data; and/or

providing an estimate for the probability of acceptable stimulation results, e.g., maximum therapeutic effect or minimal side effects or the like, based on the metadata, the positioning data and the spatial data; and/or

providing a limitation of the amount of stimulation options by preselecting possible stimulation field possibilities based on the metadata, the positioning data and the spatial data.

The method may further comprise the step of directionally displaying the areas with increased likelihood for best outcome based on the metadata, the positioning data and the spatial data, especially wherein sectors around the lead are displayed for displaying the areas with increased likelihood for best outcome based on the preselection of possible stimulation field possibilities and based on the metadata, the positioning data and the spatial data.

Also, the method may further comprise the step of simulating the effect of at least one possible stimulation field setting possibility.

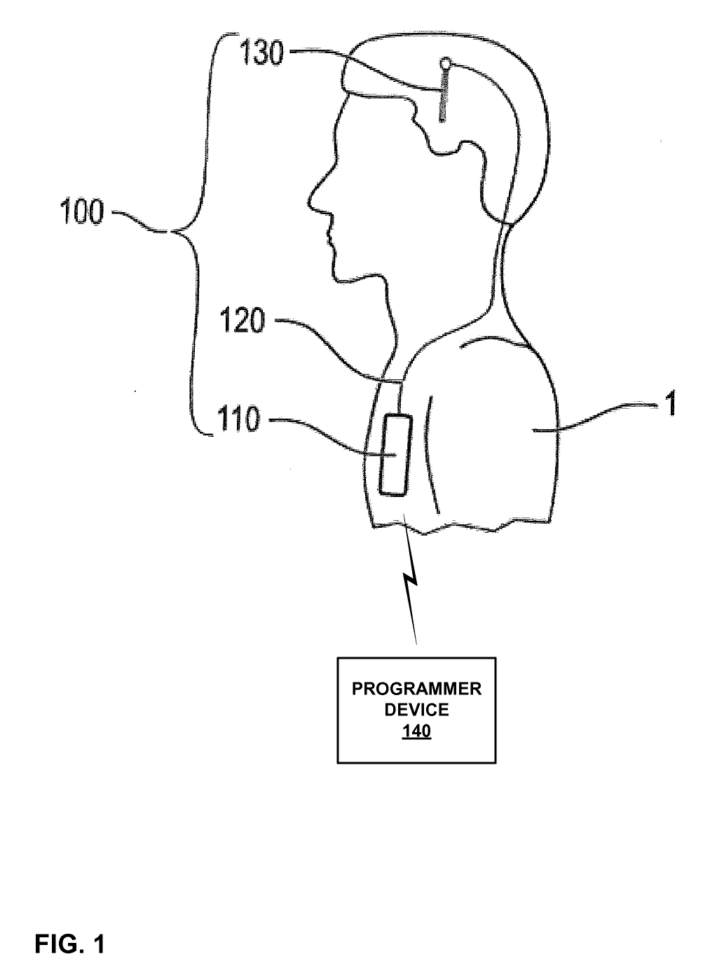

An example of a neurostimulation system 100 for deep brain stimulation (DBS) is shown in FIG. 1. The neurostimulation system 100 may comprise at least a controller 110 that may be surgically implanted in the chest region of a patient 1, typically below the clavicle or in the abdominal region of a patient 1. The controller 110 can be adapted to supply the necessary voltage or current pulses to selected electrodes on a probe 130 (also referred to as lead 130) for delivery of electrical stimulation. The typical DBS system 100 may further include an extension wire 120 (or lead extension) connected to the controller 110 and running subcutaneously to the skull, preferably along the neck, where it terminates in a connector at the distal end of the lead extension (not shown). The connector at the distal end of lead extension 120 couples to a corresponding connector at a proximal end of probe 130 (also not shown). A DBS lead arrangement, such as probe 130, may be implanted in the brain tissue, e.g., through a burr-hole in the skull. Controller 110 may also be referred to as an implantable medical device (IMD). Probe 130 may include a complex electrode geometry, such as a plurality of segmented electrodes arranged in rings at different axial positions of probe 130, and each ring comprising electrode segments at different angular positions around the probe circumference, as discussed above and further discussed below. As used herein, a segmented electrode refers to an electrode that does not extend around the entire circumference of the lead. Segmented electrodes may be any shape, such as partial rings, or some other shape. Probe 130 may also optionally include one or more full ring electrodes that extend all the way around the circumference of the lead.

System 100 may further include a programmer device 140. Programmer device 140 may generally perform certain techniques of this disclosure. In particular, a clinician or other user may interact with programmer device 140 to program controller 110. As explained in greater detail below, programmer device 140 may program controller 110 wirelessly, e.g., via wireless telemetry. Furthermore, in accordance with the techniques of this disclosure, programmer device 140 may determine an implant location of probe 130 in patient 1. Furthermore, programmer device 140 may receive scan data related to the implant location, e.g., a scan of an area of the patient's brain including the implant location. Programmer device 140 and/or one or more other processing devices that communicate with programmer device 140 may warp the scan data to match an atlas for a region including the implant location. Furthermore, based on the warped data and the atlas, programmer device 140 and/or the other processing device(s) may determine probabilities that electrical stimulation will be effective for one or more electrodes of probe 130.

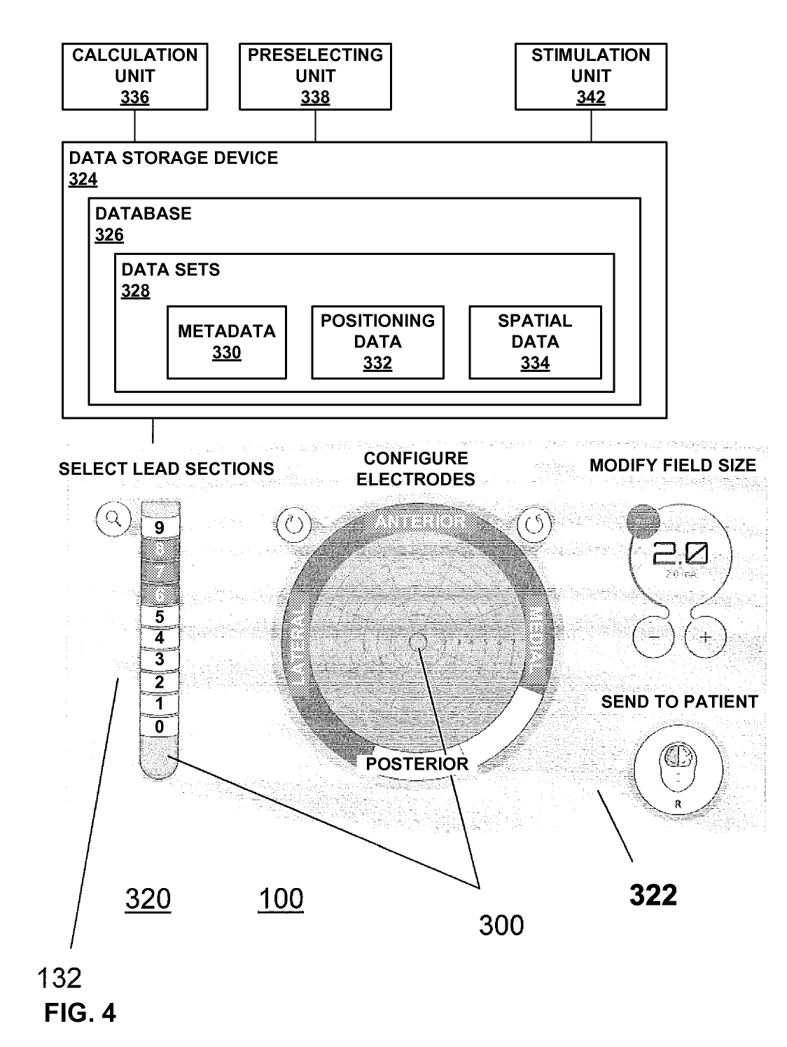

In some examples, processing steps according to the current disclosure may be performed by one or more other processing devices that may communicate with programmer device 140 and that are part of a medical system 320 as discussed below (FIG. 4). These other devices may include one or more workstations, servers, laptop computers, “cloud-based”computing systems or any other one or more systems capable of processing data. Data, such as scan data, probability data, and so on, may be transferred between programmer device 140 and one or more of these processing devices via a wired or wireless connection for use according to techniques of this disclosure. Alternatively or additionally, some processing steps may be performed by a processor of controller 110.

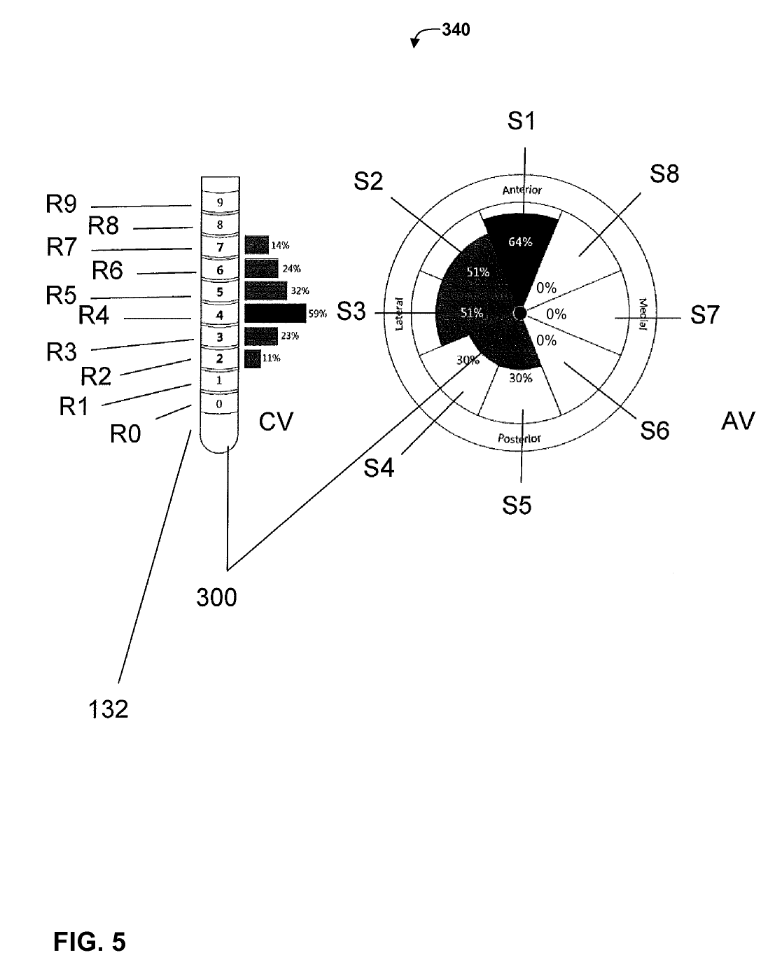

Programmer device 140 may also display a graphical representation of the determined probabilities. For example, programmer device 140 may display a graphical user interface (GUI) including a graphical representation of probe 130, e.g., a depiction of a lead, and display numeric and/or graphical representations of the probabilities. For instance, the graphical representation may include a bar chart and numeric scores for the corresponding probabilities. In some examples, probabilities may be determined for each segment of the segmented electrodes. As such, programmer device 140 may further present a graphical indication of the probabilities of effectiveness for each sector of one or more of the segmented electrodes as well. Examples of such graphical representations are shown in FIG. 5, which is discussed in greater detail below.

More particularly, historical clinical trials may be performed to generate data representative of outcomes of stimulation therapies applied to various regions of patient brains, to treat various conditions. When the clinical trials indicate that a particular region of the brain yields alleviation of a particular condition, data may be stored that is representative of the condition and the region that, when stimulated, alleviates that condition. Subsequently, when a patient presents that condition, a lead may be implanted in the patient's brain near the region (i.e., in close spatial proximity to the region). The lead may have a set of rings (e.g., segmented electrodes in the shape of a ring and/or full ring electrodes), which can be individually activated or deactivated to deliver therapeutic electrical stimulation. The rings and electrodes thereof that are closest to the region to be stimulated may be determined, and probabilities may be assigned to the electrodes based on, e.g., their spatial proximity to the region to be stimulated. In accordance with the techniques of this disclosure, values representative of probabilities of effectiveness of the electrodes may be presented to a clinician to help the clinician determine which of the electrode(s) to activate to deliver therapeutic electrical stimulation to the patient. That is, those electrodes that are likely to stimulate the brain region that has been shown to alleviate the patient's condition may be determined. For instance, electrodes that are closer to the region may be assigned higher probabilities than electrodes that are further from the region.

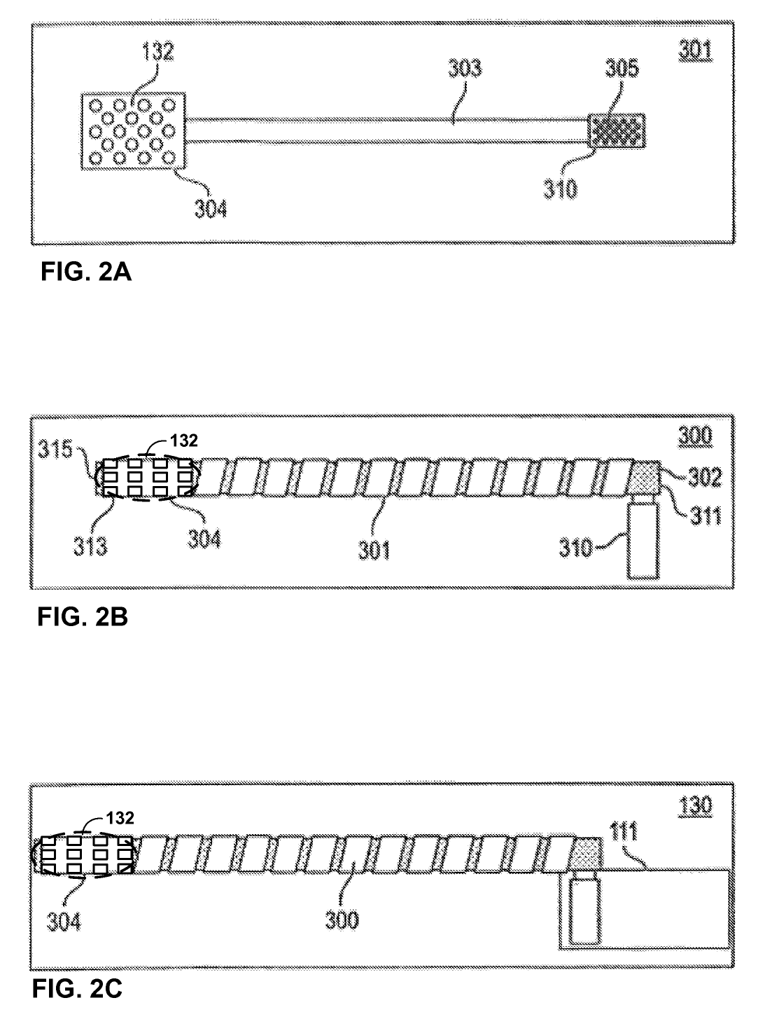

FIGS. 2A-2C further illustrate examples of typical architectures for Deep Brain Stimulation probe 130, which include a DBS lead 300. DBS lead 300 may, in some examples, include an active lead can (ALC) 111 including electronic means to address electrodes 132 on the distal end 304 of the thin film 301, which is arranged at the distal end 313 and next to the distal tip 315 of the DBS lead 300. Electrodes 132 as shown in FIGS. 2B and 2C are part of a complex electrode array of the type discussed above. As previously discussed, such electrodes may be arranged in different rings or rows around the circumference of the lead or the electrodes may be arranged in any other type of arrangement. The lead 300 comprises a carrier 302 for a thin film 301, the carrier 302 providing the mechanical configuration of the DBS lead 300 and the thin film 301. The thin film 301 may include at least one electrically conductive layer, preferably made of a biocompatible material. The thin film 301 is assembled to the carrier 302 and further processed to constitute the lead 300. The thin film 301 for a lead is preferably formed by a thin film product having a distal end 304, a cable section 303 with metal tracks and a proximal end 310. The proximal end 310 of the thin film 301 arranged at the proximal end 311 of the lead 300 is electrically connected to the active lead can 111.

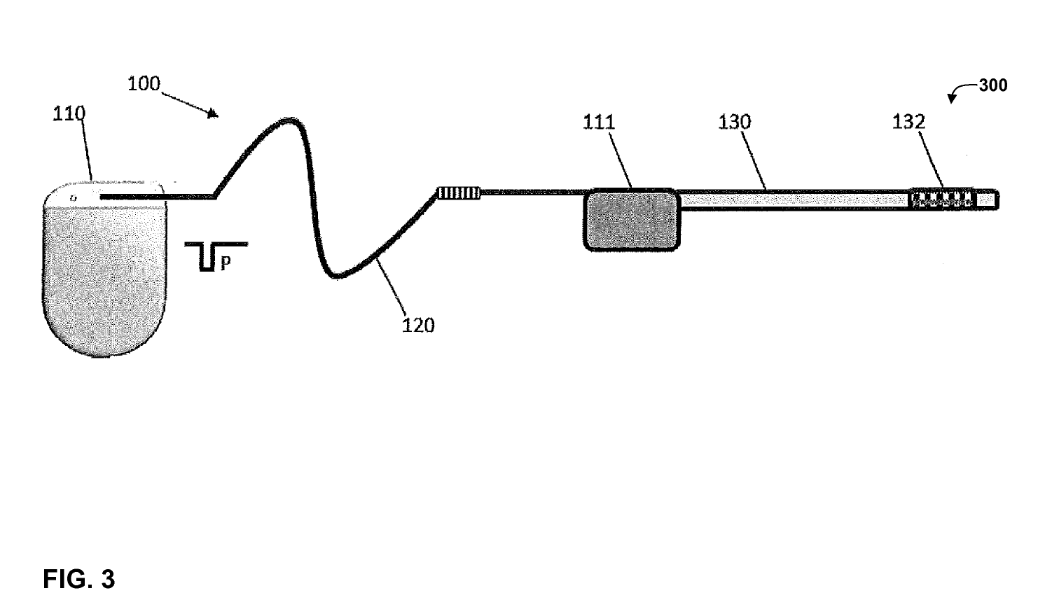

The active lead can 111 comprises the switch matrix of the DBS steering electronics. The distal end 304 comprises the electrodes 132 for the brain stimulation. The proximal end 310 comprises the interconnect contacts 305 for each conductor in the cable 303. The cable 303 comprises connectors (not shown) to connect each of distal electrodes 132 to a designated proximal contact 305, e.g., for connection to electronics in the ALC 111. In this manner, each of electrodes 132 is coupled to electronics in ALC 111. Electronics of the ALC 111 are, in turn, coupled via feedthrough connections to conductors carried by the proximal end of lead 130. A connector at the proximal end of lead 130 (FIG. 3) is adapted to couple to a counterpart connector at the distal end of lead extension 120, which is coupled to a stimulation pulse generator in controller 110 (FIG. 1).

FIG. 3 shows schematically and in greater detail an example of a system 100 for brain applications, here for neurostimulation and/or neurorecording as a deep brain stimulation system 100 as shown in FIGS. 1 and 2. The probe system 100 comprises at least one probe 130 for brain applications with stimulation and/or recording electrodes 132, wherein, e.g., 40 electrodes 132 can be provided on outer body surface at the distal end of the probe 130. By means of the extension wire 120 pulses P supplied by controller 110 can be transmitted to the active lead can 111. The controller 110 can be an implantable pulse generator 110.

FIG. 4 shows a schematic drawing of a medical system 320 with its display 322 as a part of a neural application system 100 according to the techniques of this disclosure, here a DBS system 100 as specified above, with which the techniques of this disclosure can be performed. Display 322 is discussed further below in reference to FIG. 5.

The DBS system 100 has a lead 300, wherein the lead 300 comprises a plurality of electrodes 132 forming a complex array of electrodes. For example, a plurality of rings of electrodes may be provided, where each ring may represent solid ring electrodes or segmented electrodes along the ring. Rings of segmented electrodes . . . may include, for example, eight electrodes, including anterior, posterior, medial, and lateral electrodes, and electrodes midway between each of the anterior, posterior, medial, and lateral electrodes.

The medical system 320 may comprise a tablet computer, which has a touchpad and is linked to the DBS system 100 wirelessly.

It is alternatively possible that the medical system 320 is a workstation, a stationary PC or the like, which is suitable linked the DBS system 100, either wirelessly or wired or both. The medical system may include programming device 140 discussed above.

The medical system 320 includes a suitable data storage device 324, which host a database 326 for storing a plurality of data sets 328.

The data sets comprise, inter alia, metadata 330, including at least patient data, relating to gender data, age data, local field recordings data, microelectrode recordings data, tissue fiber structures data, demographics data and/or anatomy data of a patient.

The metadata 330 may further include functional brain atlas data (FBA), like the functional brain atlas published and provided by Guo et al., 2007, Pallavaram et al., 2010, Chakravarty et al., 2006, Nowinski et al., 2004, with citations as follows:
CHAKRAVARTY, M. M., SADIKOT, A. F., MONGIA, S., BERTRAND, G. & COLLINS, D. L. 2006. Towards a multi-modal atlas for neurosurgical planning Med Image Comput Comput Assist Interv, 9, 389-96.
GUO, T., PARRENT, A. G. & PETERS, T. M. 2007. Automatic target and trajectory identification for deep brain stimulation (DBS) procedures. Med Image Comput Comput Assist Interv, 10, 483-90.
NOWINSKI, W. L., BELOV, D., POLLAK, P. & BENABID, A. L. 2004. A probabilistic functional atlas of the human subthalamic nucleus. Neuroinformatics, 2, 381-98.
PALLAVARAM, S., DAWANT, B. M., REMPLE, M. S., NEIMAT, J. S., KAO, C., KONRAD, P. E. & D'HAESE, P. F. 2010. Effect of brain shift on the creation of functional atlases for deep brain stimulation surgery. Int J Comput Assist Radiol Surg, 5, 221-8.

Furthermore, the data sets 328 may comprise positioning data 332 of an implanted neurostimulation lead 300.

Also, the data sets 328 may comprise spatial data 334 relating to the possible distribution of at least one stimulation field of the neurostimulation lead 300.

Moreover, the medical system 320 comprises calculation unit 336 for calculating possible stimulation field setting possibilities based on the metadata 330, the positioning data 332 and the spatial data 334.

Additionally, the medical system 320 comprises preselecting unit 338 for preselecting the possible stimulation field setting possibilities based on the metadata 330, the positioning data 332 and the spatial data 334.

FIG. 5 a further schematic drawing of the display 322 of the medical system 320 as shown in FIG. 4.

A Clinician Programmer User Interface 340 displayed on the display 322 currently offers a visualization of the lead 300 from a coronal view CV and an axial view AV. Also, there is a probability overview for stimulation being offered in a visually similar way to allow for quick translation and entering.

According to one example of the techniques of this disclosure, a rendering of a lead 300 may be provided with graphics and numeric values in percentage mapped to each ring R0, R1, R2, R3, R4, R5, R6, R7, R8 and R9, both to represent probability. Each ring represents several electrodes 132 out of the plurality of electrodes 132, the electrodes 132 of each ring being arranged substantially at the same axial position, i.e., same height or distance from the distal tip end of the lead 300.

The same is accomplished for directions for steering of the stimulation field generated by the plurality of electrodes 132, as shown by the axial view AV. The Clinician Programmer User Interface 340 offers resemblance to a bar chart and allows for quick assessment of preferred rings R0, R1, R2, R3, R4, R5, R6, R7, R8 and R9 and directions.

There may be a printing function, which allows the user to print out a screenshot of the currently displayed Clinician Programmer User Interface 340. For example, the system may include a user interface module communicatively coupled to a printer, such as a laser printer.

The functionality of the medical system 320, e.g., when performing the method of planning and/or tuning a neural application, here a neurostimulation therapy, i.e., a Deep Brain Stimulation Therapy, can be described as follows:

The preselecting unit 338 may select a starting point for the first stimulation from the number of preselected possible stimulation field setting possibilities by preselecting the possible stimulation field possibilities based on the metadata 330, the positioning data 332, and the spatial data 334. For example, metadata 330 may include data indicating characteristics of stimulation when applied by electrodes at various positions in various spatial arrangements. The preselecting unit 338 may, accordingly, determine the starting point for the first stimulation based on desired therapeutic effects, as well as where the lead is implanted as indicated by the positioning data 332 and the spatial data 334. The starting point may correspond to a stimulation field having the greatest probability of delivering the desired stimulation, given the position of the lead and electrodes of the lead as indicated by the positioning data 332 and the spatial data 334.

Furthermore, the preselecting unit 338 provides an estimate for the probability of acceptable stimulation results, e.g., maximum therapeutic effect or minimal side effects or the like, based on the metadata 330, the positioning data 332 and the spatial data 334.

Also, the preselecting unit 338 may provide a limitation of the amount of stimulation options by preselecting possible stimulation field possibilities based on the metadata 330, the positioning data 332 and the spatial data 334.

As shown in FIG. 4 and discussed above, the medical system 320 comprises display 322 (e.g., a user interface) for directional displaying the areas with increased likelihood for best outcome based on the metadata 330, the positioning data 332 and the spatial data 334.

Sectors S1, S2, S3, S4, S5, S6, S7 and S8 are displayed around the lead 300 for displaying the areas with increased likelihood for best outcome based on the preselection of possible stimulation field possibilities and based on the metadata 330, the positioning data 332 and the spatial data 334. In general, sectors S1-S8 represent sectors of a segmented ring electrode, where an individual electrode may be positioned within each sector.

For example, the probability for a good stimulation outcome is estimated with a probability of 64% in sector S1 of ring R_, corresponding to a particular electrode at an angular position corresponding to section S1, where “R_” refers to any of the axial positions, or rings R1-R9. That is, based on historical data and positioning of the lead and electrodes along the lead, a good stimulation outcome may correspond to a desirable stimulation effect, e.g., reduction of pain, treatment of a patient symptom, or the like. The probability in ring R_ for sector S2 is 51%, in sector S3 51%, in Sector S4 30% and in sector S5 30%.

In all other sectors, i.e., in sectors S6, S7 and S8, the probability is estimated with 0% probability of good stimulation outcome and thus the physician may immediately notice that these sectors can be omitted from consideration in establishing settings for stimulation. The 0% probability is represented in FIG. 5 by a lack of shading in the displayed regions of sectors S6, S7, and S8. In particular, the electrodes associated with the 0% sectors can be ignored, and omitted from stimulation parameter programming, as it will ordinarily be undesirable to deliver stimulation via such electrodes.

Moreover, the medical system 320 comprises simulation unit 342 for simulating the effect of at least one possible stimulation field setting possibility. Thus, the physician may conduct simulations about the outcome of stimulation settings and prepare the adjustment of the stimulation settings before adjusting the settings at the implanted lead.

In particular, with the help of the medical system and especially its preselection unit 338 using the metadata 330, i.e., functional brain atlas data, the positioning data 332 and the spatial data 334, clinicians can be assisted or given guidance during programming of the stimulation settings by providing for the following:
1. A starting point for the first stimulation.
2. A probability of acceptable stimulation results (i.e., maximum therapeutic effects, minimal side effects).
3. A limitation of the amount of options.

Together, these additions may decrease the amount of time required to perform programming. Furthermore, a disadvantage in the current practice is that it requires effort to translate conclusions drawn in procedural steps prior to tuning into usable input for tuning. This may include, inter alia, suggestions on which rings of electrodes/level of electrodes stimulation is to be provided, as concluded during intra-operative recording and placement and any conclusions drawn from a pre-analysis of the stimulation possibilities. The outcome of the preselection done by the medical system is offered to the clinician in a format that allows him to use it quickly and practically during a programming session.

One example of the techniques of this disclosure is to provide an appearance model of the lead 300 with individually distinguishable rings in a digital environment. Each ring would be paired with a visualization that signifies the probability of desired stimulation results if that ring were configured to be active (e.g., anodic or cathodic). In addition, specific azimuthal directions can similarly be paired with a visualization indicating probability. The image will also be printable to regular paper by a regular (consumer) printer. This way it can be used as reference material during the programming session in the Clinician Programmer Tuning software.

In this manner, the system may present a representation of one or more electrodes and a representation of probabilities of the electrodes delivering effective therapy to a patient, e.g., based on images of the patient's anatomy and an implant location of a lead including the electrodes. Furthermore, as shown in the example of FIG. 5, a first representation may depict probabilities that individual rings of segmented electrodes (that is, segmented ring electrodes) in a set of such rings along a lead will deliver effective therapy, and a second representation of probabilities that individual electrodes of one of the rings will deliver effective therapy. For instance, as portrayed in the example of FIG. 5, ring R4 has the highest probability of delivering effective therapy, and the electrode corresponding to sector S1 of ring R4 has the highest probability of delivering effective therapy.

It may be noted that a first and second representation according to this disclosure could include any number of axial levels (rings) and any number of sectors, respectively, depending on the configuration of the lead. Furthermore, if the lead includes staggered electrodes, wherein not all electrodes are aligned in the same longitudinal columns (similar to as shown in FIGS. 2A-2B, the electrodes of one rings may be in sectors that are different from the electrodes of another ring. For instance, the rings at odd locations may include electrodes in odd sectors, whereas rings at even locations may include electrodes in even sectors.

Visualizing probabilities of good stimulation results for rings and directions in such a manner may yield the following benefits during programming:
Assessing the outcomes of prior analysis more quickly and effectively.
Potentially significantly lowering the amount of combinations the clinician has to test.
Simplification of creating configurations for stimulation if the software used for tuning offers visual resemblance of that lead model (see FIGS. 4 and 5).
Allowing the visualization to be printed eases transfer or communication of settings between different clinicians or software applications.

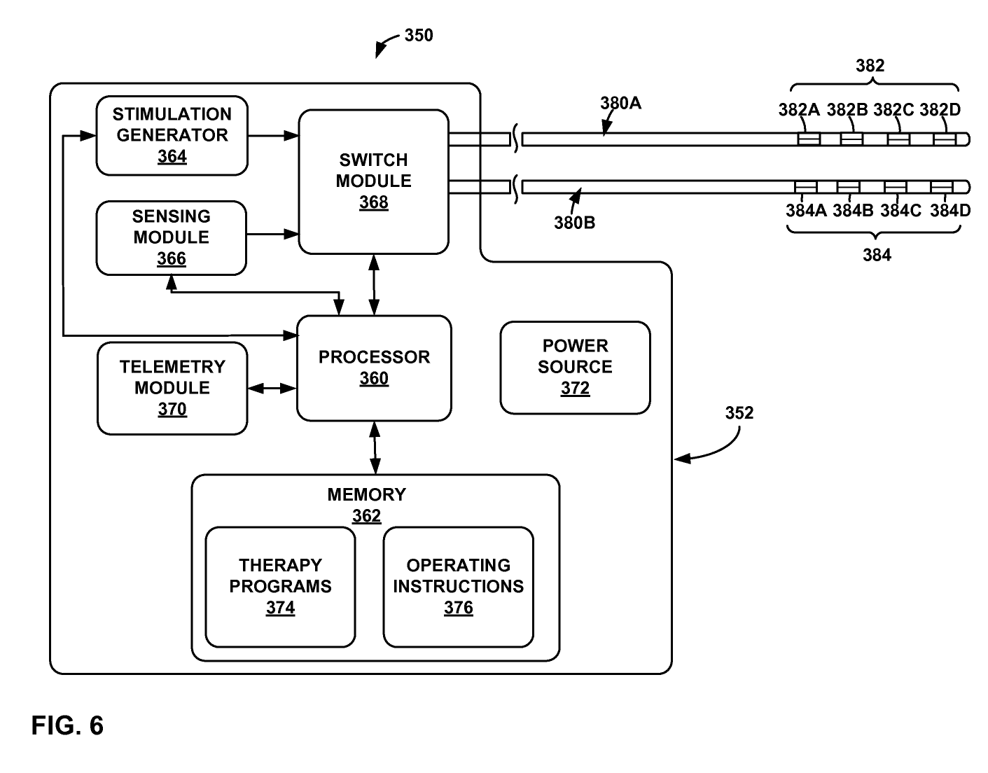

FIG. 6 is a block diagram illustrating IMD 350 and leads 380A and 380B. IMD 350 and leads 380 may generally correspond to controller 110, and probe (or lead) 130 of FIGS. 1-5. In a manner similar to that shown in FIG. 1 with lead extension 120, in some examples, a lead extension may be coupled between IMD 350 and the leads 380, however, this is not shown in FIG. 6. Additionally, in some examples such as that shown in FIG. 1, an ALC 111 may be carried by one or more of leads 380, although this is not shown in FIG. 6. As discussed above, the ALC 111 may include electronics such as a switch matrix to select electrodes of the lead that are to provide the stimulation to the patient.

In the example shown in FIG. 6, IMD 350 includes processor 360, memory 362, stimulation generator 364, sensing module 366, switch module 368, telemetry module 370, and power source 372. Memory 362, as well as other memories described herein, may include any volatile or non-volatile media, such as a random access memory (RAM), read only memory (ROM), non-volatile RAM (NVRAM), electrically erasable programmable ROM (EEPROM), flash memory, and the like. Memory 362 may store computer-readable instructions that, when executed by processor 360, cause IMD 350 to perform various functions described herein.

In the example shown in FIG. 6, memory 362 stores therapy programs 374 and operating instructions 376, e.g., in separate memories within memory 362 or separate areas within memory 362. Each stored therapy program 374 defines a particular program of therapy in terms of respective values for electrical stimulation parameters, such as an electrode combination, current or voltage amplitude, and, if stimulation generator 364 generates and delivers stimulation pulses, the therapy programs may define values for a pulse width, and pulse rate of a stimulation signal. Each stored therapy program 374 may also be referred to as a set of stimulation parameter values. Operating instructions 376 guide general operation of IMD 350 under control of processor 360, and may include instructions for monitoring brain signals within one or more brain regions via segmented electrodes 382, 384 and delivering electrical stimulation therapy to the patient. Segmented electrodes 382, 384 may correspond to segmented electrodes such as those shown as electrodes 132 of FIGS. 2-5. In other examples, full ring electrodes that extend all the way around the circumference of the lead may be used in place of, or in addition to, segmented electrodes 382. That is, one or more of segmented electrodes 382 may be replaced by ring electrodes, and/or additional electrodes may be added in addition to electrodes 382 as shown.

Stimulation generator 364, under the control of processor 360, generates stimulation signals for delivery to the patient via selected combinations of electrodes 382, 384 In some examples, stimulation generator 364 generates and delivers stimulation signals to one or more target regions of the patient's brain, via a selected combination of electrodes 382, 384, based on one or more stored therapy programs 374. The target tissue sites within the patient's brain for stimulation signals or other types of therapy and stimulation parameter values may depend on the patient condition for which therapy system 100 is implemented to manage. While stimulation pulses are described, stimulation signals may be of any form, such as continuous-time signals (e.g., sine waves) or the like. In some examples, stimulation generator may have multiple channels that are capable of delivering independent signals to one or more of the electrodes at the same time.

The processors described in this disclosure, including processor 360, may include one or more digital signal processors (DSPs), general purpose microprocessors, application specific integrated circuits (ASICs), field programmable logic arrays (FPGAs), or other equivalent integrated or discrete logic circuitry, or combinations thereof. The functions attributed to processors described herein may be provided by a hardware device and embodied as software, firmware, hardware, or any combination thereof. Processor 360 is configured to control stimulation generator 364 according to therapy programs 374 stored by memory 362 to apply particular stimulation parameter values specified by one or more programs, such as amplitude, pulse width, and pulse rate.

In the example shown in FIG. 6, the set of segmented ring electrodes 382 of lead 380A includes segmented ring electrodes 382A, 382B, 382C, and 382D, and the set of segmented ring electrodes 384 of lead 380B includes electrodes 384A, 384B, 384C, and 384D. Processor 360 may control switch module 368 to apply the stimulation signals generated by stimulation generator 364 to selected electrodes or combinations of electrodes of electrodes 382, 384. That is, after a clinician or other user determines one or more electrodes of electrodes 382, 384 that have a high likelihood of delivering effective therapy, the clinician may program IMD 350 to deliver therapy accordingly. For example, the clinician may select the electrode (e.g., a particular one of electrodes 382, 384 and one of the electrodes of the segmented ring electrodes), or combinations of such electrodes, to deliver therapy that has the highest probability of delivering effective therapy (that is, that has the highest probability of achieving a desired therapeutic outcome, and program IMD 350 to deliver stimulation via the one (or more) determined electrodes.

Switch module 368 may couple stimulation signals to selected conductors within leads 380, which, in turn, deliver the stimulation signals across selected segmented ring electrodes 382, 384. Switch module 368 may be a switch array, switch matrix, multiplexer, or any other type of switching module configured to selectively couple stimulation energy to selected electrodes 382, 384 and to selectively sense bioelectrical brain signals with selected electrodes 382, 384. Hence, stimulation generator 364 is coupled to electrodes 382, 384 via switch module 368 and conductors within leads 380. Each electrode may be individually addressable in some examples. In some examples, however, IMD 350 does not include switch module 368.

Stimulation generator 364 may be a single channel or multi-channel stimulation generator. In particular, stimulation generator 364 may be configured to delivering a single stimulation pulse, multiple stimulation pulses or continuous signal at a given time via a single electrode combination or multiple stimulation pulses at a given time via multiple electrode combinations. In some examples, however, stimulation generator 364 and switch module 368 may be configured to deliver multiple channels on a time-interleaved basis. For example, switch module 368 may serve to time divide the output of stimulation generator 364 across different electrode combinations at different times to deliver multiple programs or channels of stimulation energy to the patient. Sensing module 366, under the control of processor 360, is configured to sense bioelectrical brain signals of the patient via a selected subset of segmented ring electrodes 382, 384 or with one or more segmented ring electrodes 382, 384 and at least a portion of a conductive outer housing 352 of IMD 350, an electrode on an outer housing of IMD 350 or another reference. Processor 360 may control switch module 368 to electrically connect sensing module 366 to selected segmented ring electrodes 382, 384. In this way, sensing module 366 may selectively sense bioelectrical brain signals with different combinations of segmented ring electrodes 382, 384 (and/or a reference other than an electrode 382, 384).

Although sensing module 366 is incorporated into a common housing 352 with stimulation generator 364 and processor 360 in FIG. 6, in other examples, sensing module 366 is in a separate outer housing from outer housing 352 of IMD 350 and communicates with processor 360 via wired or wireless communication techniques.

Telemetry module 370 is configured to support wireless communication between IMD 350 and an external programmer device 400 or another computing device under the control of processor 360. Processor 360 of IMD 350 may receive, as updates to programs, values for various stimulation parameters such as amplitude and electrode combination, from programmer device 400 via telemetry module 370. The updates to the therapy programs may be stored within therapy programs 374 portion of memory 362. Telemetry module 370 in IMD 350, as well as telemetry modules in other devices and systems described herein, such as programmer device 400, may accomplish communication by RF communication techniques. In addition, telemetry module 370 may communicate with external medical device programmer device 400 via proximal inductive interaction of IMD 350 with programmer device 400. Accordingly, telemetry module 370 may send information to external programmer device 400 on a continuous basis, at periodic intervals, or upon request from IMD 350 or programmer device 400.

Power source 372 delivers operating power to various components of IMD 350. Power source 372 may include a small rechargeable or non-rechargeable battery and a power generation circuit to produce the operating power. Recharging may be accomplished through proximal inductive interaction between an external charger and an inductive charging coil within IMD 350. In some examples, power requirements may be small enough to allow IMD 350 to utilize patient motion and implement a kinetic energy-scavenging device to trickle charge a rechargeable battery. In other examples, traditional batteries may be used for a limited period of time.

FIG. 7 is a functional block diagram illustrating components of an example medical device programmer device 400. Programmer device 400 may correspond to, for example, programmer device 140 (FIG. 1) or to medical system 320 (FIG. 4). Programmer device 400 includes processor 402, memory 404, telemetry module 406, user interface 408, and power source 410. Processor 402 controls user interface 408 and telemetry module 406, and stores and retrieves information and instructions to and from memory 404. Programmer device 400 may be configured for use as a clinician programmer or a patient programmer. Processor 402 may comprise any combination of one or more processors including one or more microprocessors, DSPs, ASICs, FPGAs, or other equivalent integrated or discrete logic circuitry. Accordingly, processor 402 may include any suitable structure, whether in hardware, software, firmware, or any combination thereof, to perform the functions ascribed herein to processor 402.

A user, such as a clinician or the patient, may interact with programmer device 400 through user interface 408. User interface 408 includes a display (not shown), such as a LCD or LED display or other type of screen, with which processor 402 may present information related to the therapy (e.g., electrodes and associated therapeutic windows). In addition, user interface 408 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces, such as display 322 of FIGS. 4 and 5, presented by processor 402 of programmer device 400 and provide input. In other examples, user interface 408 also includes audio circuitry for providing audible notifications, instructions or other sounds to the patient, receiving voice commands from the patient, or both.

Memory 404 may include instructions for operating user interface 408 and telemetry module 406, and for managing power source 410. In the example shown in FIG. 7, memory 404 also stores regions 412, patient anatomy data 414, therapy programs 416, and efficacy map information 418.

Regions 412 stores information identifying one or more regions of tissue of the patient's brain (or another part of the body of the patient) associated with efficacious therapy delivery. These regions may be referred to as efficacy regions. Regions 412 also stores information identifying one or more regions of tissue of the patient's brain (or another part of the body of patient) associated with adverse stimulation effects. These regions may be referred to as adverse-effects regions. The regions 412 may be identified using any suitable convention. In some examples, the efficacy and adverse-effects regions are identified by specific brain structures or parts of brain structures, coordinates of any suitable coordinate system to which leads 380 and the patient's brain are registered, other anatomical structures, pixels of a two-dimensional (2D) grid to which the patient's brain or another portion of the body of the patient is registered, voxels of a three-dimensional (3D) grid to which the patient's brain or another portion of the body of the patient is registered (as discussed in further detail below), or any combination thereof.

The efficacy regions and adverse-effects regions stored by regions 412 may differ depending on the patient condition. For example, if therapy system 100 is implemented to manage tremors experienced by the patient, regions 412 may include the substantia nigra because for some patients, stimulating the substantia nigra may help reduce the number and magnitude of tremors experienced by the patient.

In some examples, a clinician selects the stored regions 412. In other examples, the regions 412 are preselected and associated with a patient condition; processor 402 or a clinician may determine the regions 412 relevant to the patient by selecting the patient condition for which system 100 is implemented to manage.

In some examples, processor 402 is configured to store determined efficacy maps in memory 404 as efficacy map information 418. A clinician may review the stored efficacy map information 418, e.g., during programming of IMD 350 to select one or more of therapy programs 416 by which IMD 350 may deliver efficacious electrical stimulation to the patient. For example, the clinician may interact with user interface 408 to retrieve the stored efficacy map information 418.

In accordance with the techniques of this disclosure, memory 404 also stores atlas information 420. Atlas information 420 may include one or more atlases, generally formed from a plurality of previously studied brain anatomies to form a general purpose model. Efficacy map information 418 may be substantially associated with atlas information 420, where efficacy map information 418 may indicate areas of the atlases that can be stimulated electrically via electrodes to yield a desired outcome. Thus, processor 402 may geometrically warp data for a particular patient's anatomy to the atlas, such that a graphical representation of a lead as implanted in the patient is represented within a graphical representation of the atlas. Then, processor 402 may determine which of the various electrodes of the lead is closest to an area of the atlas that can be stimulated for a particular desired outcome. From this determination, processor 402 may determine probabilities that each of the various electrodes will effectively deliver electrical stimulation for the patient.

For example, suppose that an electrode in a lateral sector of ring R5 of an electrode is closest to a stimulation zone that should yield a desired therapeutic outcome via electrical stimulation. Processor 402 may determine that an electrode in the lateral sector of ring R5 has a highest probability of delivering effective electrical stimulation, that similar sectors of rings 4 and 6 have electrodes with the next lower probabilities of delivering effective electrical stimulation, and that similar sectors of rings 3 and 7 have electrodes with the lowest, non-zero probabilities of delivering effective electrical stimulation. Accordingly, processor 402 may cause a display of user interface 408 to present a GUI representative of these determinations. The GUI may resemble display 322 of FIGS. 4 and 5, in some examples. In general, metadata 330 may include definitions of the probabilities and/or formulas for calculating the probabilities based on positioning of the lead (and electrodes along the lead).

Although activation of single electrodes for delivery of stimulation, e.g., in a unipolar configuration with an electrode on an IPG case as a return electrode, is discussed above for purposes of example, it should be understood that in other examples, other electrode configurations may be analyzed. For example, the techniques of this disclosure may be applied when two or more electrodes are activated in an array of active electrodes, e.g., in a bipolar or multipolar configuration. For example, one or more electrodes may be configured as anodes and one or more other electrodes may be configured as cathodes. The techniques of this disclosure may be used to present graphical representations of probabilities that certain electrodes or combinations of electrodes will be effective when delivering electrical stimulation, thereby guiding a clinician in selecting electrodes and setting stimulation parameters for the selected electrodes.

Based on the display, a user (such as a clinician) may determine probabilities that certain electrodes (or combinations of electrodes) will be effective when used to deliver electrical stimulation. In particular, the clinician may determine the probabilities of effectiveness prior to actually delivering any therapeutic electrical stimulation to the patient. In this manner, the techniques of this disclosure may reduce an amount of time between implantation of an IMD and configuration of the IMD to deliver the most effective therapy for the patient.

In some examples, the clinician may select a most probable electrode or electrode combination, in terms of probability of good stimulation result, to be used to deliver therapy to the patient. In other examples, the clinician may develop more than one therapy program, such that two or more of the most probable electrodes or electrode combinations are included in a list of potential therapy programs. In such examples, the clinician may program IMD 350 to operate according to each of the various programs, e.g., at various times of day or according to patient input (e.g., via a patient programmer) to cycle through the list of programs to identify a most effective therapy program in the list for the patient.

In some examples, the clinician (or another user) may provide input via user interface 408 to manipulate a score, an efficacy score, a clinical rating scale score, or efficacy map information. For example, in response to receiving user input requesting the list of therapy programs be ordered by efficacy score, or estimated clinical rating scale score, processor 402 may reorganize the list of electrodes based on the efficacy score, the estimated clinical rating scale score, or a combination thereof (e.g., from large to small or vice versa). In some examples, the clinician may update efficacy map information 418 based on patient feedback.

Processor 402 may be configured to generate other types of interfaces. For example, processor 402 may be configured to generate a display including a list of a plurality electrodes combinations (e.g., each electrode combination may be assigned a unique alphanumeric identifier or a graphical identifier) ordered based on the associated efficacy scores or estimated clinical ratings scores without displaying the associated efficacy score or estimated clinical rating scale score. For example, the five electrode combinations with the highest associated efficacy score or estimated clinical rating scale score may be displayed. The clinician may then provide input via user interface 408 requesting additional information about a particular electrode combination. In response to receiving the user input, processor 402 may present another user interface with further details about the selected electrode combination, an efficacy score, an estimated clinical rating scale score, or a plurality of subscores.

In some examples, the patient, a clinician or another user may interact with user interface 408 of programmer device 400 in other ways to manually select therapy programs, generate new therapy programs, modify therapy programs, transmit the new programs to IMD 350, or any combination thereof.

Memory 404 may include any volatile or nonvolatile memory, such as RAM, ROM, EEPROM or flash memory. Memory 404 may also include a removable memory portion that may be used to provide memory updates or increases in memory capacities. A removable memory may also allow sensitive patient data to be removed before programmer device 400 is used by a different patient.

Wireless telemetry in programmer device 400 may be accomplished by RF communication or proximal inductive interaction of external programmer device 400 with IMD 350. This wireless communication is possible through the use of telemetry module 406. Accordingly, telemetry module 406 may be similar to the telemetry module contained within IMD 350. In other examples, programmer device 400 may be configured to infrared communication or direct communication through a wired connection. In this manner, other external devices may be configured to communicating with programmer device 400 without needing to establish a secure wireless connection.

Power source 410 is configured to deliver operating power to the components of programmer device 400. Power source 410 may include a battery and a power generation circuit to produce the operating power. In some examples, the battery may be rechargeable to allow extended operation. Recharging may be accomplished by electrically coupling power source 410 to a cradle or plug that is connected to an alternating current (AC) outlet. In addition, recharging may be accomplished through proximal inductive interaction between an external charger and an inductive charging coil within programmer device 400. In other examples, traditional batteries (e.g., nickel cadmium or lithium ion batteries) may be used. In addition, programmer device 400 may be directly coupled to an alternating current outlet to operate.

Efficacy map information 418 may store one or more efficacy maps. In some examples, each efficacy map stored in efficacy map 418 may be associated with a different patient condition or symptom. In some examples, the efficacy maps may be clinical rating scale score efficacy maps (CRSEM), each associated with a particular patient condition or symptom. For example, efficacy map information 418 may include a CRSEM for each of a variety of clinical rating scales such as UPDRS, YBOCS and HDRS. The CRSEM information may be used to identify functional locations with the patient's brain. For example, the CRSEM information may include information regarding which areas of the brain provide the most therapeutic effect when activated for a particular condition. This information may be, for example, values associated with particular voxels within the CRSEM. In some examples, regions 412 may be taken into consideration when selecting a therapy program based on a CRSEM stored in efficacy map information 418 associated with a particular patient condition in scoring a therapy program.

While various information is shown stored in memory 404 of programmer device 400, it should be understood that some or all of this information could alternatively or additionally be stored within memory 362 of IMD 350. As merely one example, raw or encoded patient anatomy data 414 may be stored within memory 362 of IMD 350 for portability. Moreover, at least some of the functionality ascribed to processor 402 of programmer device 400 may instead or additionally be ascribed to processor 360 of IMD.

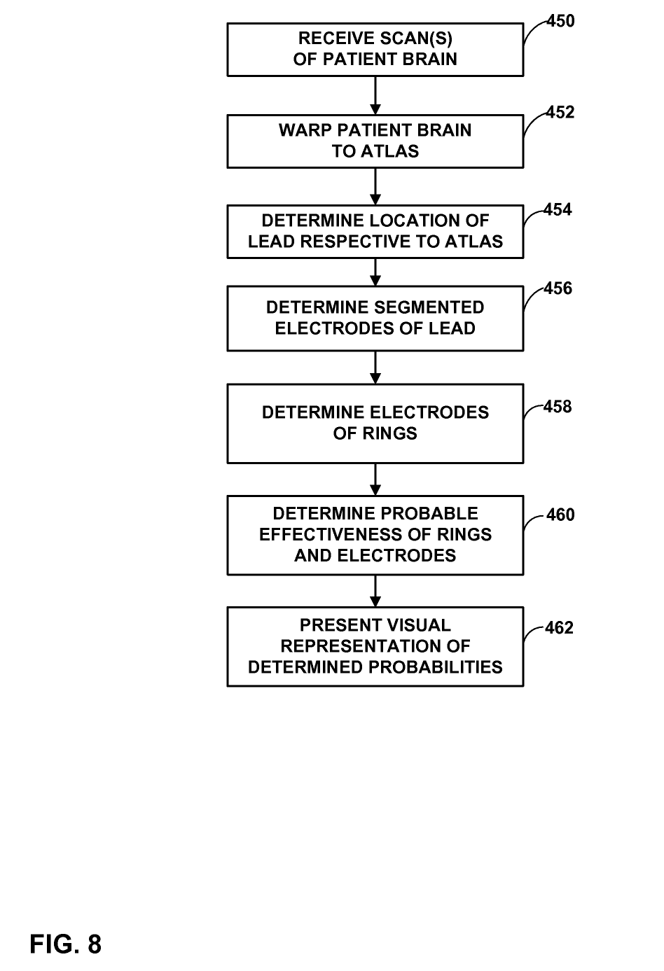

FIG. 8 is a flowchart illustrating an example method that may be used when performing the techniques of this disclosure. For purposes of explanation, FIG. 8 is described with respect to programmer device 400 of FIG. 7, although it should be understood that other devices (such as programmer device 140 or medical system 320) may be configured to perform the method of FIG. 8.

Initially, programmer device 400 may receive one or more scans of a patient brain (450). The scans may include, for example, MRI or fMRI scans of the patient's brain and a CT scan to identify the location of an implanted lead in the patient's brain. Programmer device 400 may warp the representation of the patient's brain from the received scans to an atlas (452). For example, programmer device 400 may apply spatial transformation functions to the images of the individual patient's brain such that the patient's brain substantially overlaps with the atlas. From this warped representation, programmer device 400 may determine a location of the lead respective to the atlas (454).

Programmer device 400 may then determine positions of rings of electrodes along the lead (456), as well as positions of electrodes of the rings of electrodes around the circumference of the lead (458). Based on the positions of these electrodes, programmer device 400 may determine probable effectiveness of the rings and the electrodes of the rings (460). For example, as discussed above, programmer device 400 may determine positions of the various sectors relative to a desired stimulation zone of the patient's brain. The desired stimulation zone generally corresponds to a portion of the patient's brain near the implanted lead that has been shown through historical clinical trials to yield desirable therapeutic outcomes when electrically stimulated. In general, programmer device 400 may assign higher probabilities to electrodes that are closest to the stimulation zone and lower probabilities to electrodes that are further from the stimulation zone. Moreover, programmer device 400 may reduce probabilities of electrodes that may stimulate undesirable areas of the patient's brain, e.g., by setting probabilities for such electrodes to zero.

Programmer device 400 may then present a visual (i.e., a graphical) representation of the determined probabilities (462). For example, programmer device 400 may present a first graphical representation of probabilities for each ring, or row, of the lead. The graphical representations may include numeric and/or graphical representations, such as numbers representative of the probabilities and/or graphical bars. These representations may be displayed in close spatial proximity to a graphical representation of the lead and electrodes thereof. For instance, the graphical bars and/or numeric scores may be displayed next to the corresponding rings of the displayed lead, e.g., as shown in FIG. 5. Likewise, programmer device 400 may present a second graphical representation of probabilities for each sector of one or more of the rings. For example, the second graphical representation may correspond to sectors of the ring having the highest probability or to a ring selected by a user, e.g., the clinician. In response to selection of a different ring, programmer device 400 may update the second graphical representation to reflect probabilities for sectors of the newly selected ring. Again, an example of such a second graphical representation is shown in FIG. 5.

Although not shown in FIG. 8, the method may further include receiving a selection of one or more electrodes to be used when delivering therapeutic electrical stimulation to the patient, e.g., from a clinician via programmer device 400. Programmer device 400 may further receive clinician input to program an IMD implanted in the patient to deliver therapy according to these selections, and with various stimulation parameters specified by the clinician.

In this manner, the method of FIG. 8 represents an example of a method including determining an implant location of a lead in a patient, wherein the lead comprises a plurality of electrodes, determining, based on the implant location, probabilities of effectiveness of electrical stimulation delivered via each of the electrodes, and presenting a visual representation of the determined probabilities.

The techniques described in this disclosure may be implemented, at least in part, in hardware, software, firmware or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

While this disclosure primarily describes techniques in regards to deep brain stimulation therapy, these techniques may be applied to systems and methods of treating patients suffering from a variety of conditions, such as chronic pain, tremor, Parkinson's disease, epilepsy, urinary or fecal incontinence, sexual dysfunction, obesity, gastroparesis, and cardiac disease. As examples, electrical stimulation generators are used for chronic delivery of electrical stimulation therapies such as cardiac pacing, neurostimulation, peripheral nerve stimulation, peripheral nerve field stimulation, muscle stimulation, or the like. The techniques described in this disclosure may also be embodied or encoded in a computer-readable medium, such as a computer-readable storage medium, containing instructions. Instructions embedded or encoded in a computer-readable medium may cause a programmable processor, or other processor, to perform the method, e.g., when the instructions are executed. Computer-readable media may include non-transitory computer-readable storage media and transient communication media. Computer readable storage media, which is tangible and non-transitory, may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer-readable storage media. It should be understood that the term “computer-readable storage media” refers to physical storage media, and not signals, carrier waves, or other transient media.

Various examples have been described. These and other examples are within the scope of the following claims.

What is claimed is:

1. A method of selecting one or more parameters for electrical stimulation therapy, the method comprising:
determining, by one or more processors, based on an implant location of a lead in a patient, wherein the lead comprises a plurality of electrodes, probabilities of effectiveness of electrical stimulation delivered via each of the electrodes;
presenting, by the one or more processors, a visual representation of the determined probabilities; and
in response to presenting the visual representation, receiving, by the one or more processors user input specifying one or more parameters to control delivery of electrical stimulation by an electrical stimulator to the patient.

2. The method of claim 1, wherein presenting comprises presenting the visual representation of the determined probabilities with a visual representation of the lead and the electrodes.

3. The method of claim 1, wherein the electrodes comprise electrodes of a plurality of segmented electrodes, and wherein determining the probabilities comprises determining probabilities of effectiveness for each of the electrodes of each of the segmented electrodes.

4. The method of claim 3, wherein presenting comprises:
presenting a first visual representation of the determined probabilities for each of the segmented electrodes; and
presenting a second visual representation of the determined probabilities for the electrodes of at least one of the segmented electrodes.

5. The method of claim 3, wherein the electrodes comprise at least one full ring electrode, and wherein determining the probabilities comprises determining a probability of effectiveness for each of the at least one full ring electrode.

6. The method of claim 3, wherein the electrodes comprise a first ring electrode at a proximal end of the lead, a second ring electrode at a distal end of the lead, and two segmented electrodes between the first ring electrode and the second ring electrode.

7. The method of claim 6, wherein the two segmented electrodes each include three electrodes.

8. The method of claim 1,
wherein determining the probabilities comprises determining probabilities for combinations of the electrodes; and
wherein presenting comprises presenting the visual representation to represent the probabilities for the combinations of the electrodes.

9. The method of claim 1, wherein determining the probabilities of effectiveness comprises determining the probabilities of effectiveness prior to delivering electrical stimulation to the patient via the electrodes.

10. The method of claim 1, wherein determining the probabilities of effectiveness comprises:
obtaining one or more images of the implant location (?), wherein each of the images represents one or more anatomical features of the patient near the implant location;
warping one or more of the anatomical features to fit one or more atlases for the anatomical features; and
determining the probabilities of effectiveness based at least in part on the warped anatomical features and the one or more atlases.

11. The method of claim 10, wherein the one or more anatomical features comprise a brain implant site of the patient, and wherein the one or more atlases comprise a brain implant site atlas corresponding to the brain implant site of the patient.

12. The method of claim 1, wherein determining the probabilities of effectiveness comprises determining the probabilities of effectiveness based at least in part on proximity or directionality of the electrodes to selected anatomical features of the patient.

13. A computing device for selecting one or more parameters for electrical stimulation therapy, the computing device comprising:
a memory to store data representative of an implant location of a lead in a patient, wherein the lead comprises a plurality of electrodes;
a user interface; and
one or more processors configured to:
determine, based on the implant location, probabilities of effectiveness of electrical stimulation delivered via each of the electrodes,
present, via the user interface, a visual representation of the determined probabilities, and
receive, via the user interface, user input specifying one or more parameters to control delivery of electrical stimulation by an electrical stimulator to the patient.

14. The computing device of claim 13, wherein the one or more processors are configured to present the visual representation of the determined probabilities with a visual representation of the lead and the electrodes.

15. The computing device of claim 13, wherein the electrodes comprise electrodes of a plurality of segmented ring electrodes, and wherein the one or more processors are configured to determine probabilities of effectiveness for each of the electrodes of each of the segmented rings.

16. The computing device of claim 15, wherein the one or more processors are configured to:
present a first visual representation of the determined probabilities for each of the segmented ring electrodes; and
present a second visual representation of the determined probabilities for the electrodes of at least one of the segmented ring electrodes.

17. The computing device of claim 15, wherein the electrodes comprise at least one full ring electrode, and wherein the one or more processors are configured to determine probabilities of effectiveness for each of the at least one full ring electrode.

18. The computing device of claim 13, wherein the one or more processors are configured to determine the probabilities for combinations of the electrodes, and to present the visual representation to represent the probabilities for the combinations of the electrodes.

19. The computing device of claim 13, wherein determining the probabilities of effectiveness comprises determining the probabilities of effectiveness prior to delivering electrical stimulation to the patient via the electrodes.

20. The computing device of claim 13, wherein to determine the probabilities of effectiveness, the one or more processors are configured to:
obtain one or more images of the implant location (?), wherein each of the images represents one or more anatomical features of the patient near the implant location;
warp one or more of the anatomical features to fit one or more atlases for the anatomical features; and
determine the probabilities of effectiveness based at least in part on the warped anatomical features and the one or more atlases.

21. The computing device of claim 20, wherein the one or more anatomical features comprise a brain implant site of the patient, and wherein the one or more atlases comprise a brain implant site atlas corresponding to the brain implant site of the patient.

22. A computer-readable storage medium having stored thereon instructions that, when executed, cause a processor to:
determine, based on an implant location of a lead in a patient, wherein the lead comprises a plurality of electrodes, probabilities of effectiveness of electrical stimulation delivered via each of the electrodes;
present a visual representation of the determined probabilities; and
in response to presenting the visual representation, receive user input specifying one or more parameters to control delivery of electrical stimulation by an electrical stimulator to the patient.

23. The computer-readable storage medium of claim 22, wherein the instructions that cause the processor to present comprise instructions that cause the processor to present the visual representation of the determined probabilities with a visual representation of the lead and the electrodes.

24. The computer-readable storage medium of claim 22, wherein the electrodes comprise electrodes of a plurality of segmented ring electrodes, and wherein the instructions that cause the processor to determine the probabilities comprise instructions that cause the processor to determine probabilities of effectiveness for each of the electrodes of each of the segmented ring electrodes.

25. The computer-readable storage medium of claim 24, wherein the instructions that cause the processor to present comprise instructions that cause the processor to:
present a first visual representation of the determined probabilities for each of the segmented ring electrodes; and
present a second visual representation of the determined probabilities for the electrodes of at least one of the segmented ring electrodes.

26. The computer-readable storage medium of claim 22,
wherein the instructions that cause the processor to determine the probabilities comprise instructions that cause the processor to determine probabilities for combinations of the electrodes; and
wherein the instructions that cause the processor to present comprise instructions that cause the processor to present the visual representation to represent the probabilities for the combinations of the electrodes.

27. The computer-readable storage medium of claim 22, wherein the instructions that cause the processor to determine the probabilities of effectiveness comprise instructions that cause the processor to determine the probabilities of effectiveness prior to delivery of electrical stimulation to the patient via the electrodes.

28. The computer-readable storage medium of claim 22, wherein the instructions that cause the processor to determine the probabilities of effectiveness comprise instructions that cause the processor to:
obtain one or more scans of the implant location the patient, wherein the scan represents one or more anatomical features of the patient near the implant location;
warp one or more of the anatomical features to fit one or more atlases for the anatomical features; and
determine the probabilities of effectiveness based at least in part on the warped anatomical features and the one or more atlases.

29. The computer-readable storage medium of claim 28, wherein the one or more anatomical features comprise a brain implant site of the patient, and wherein the one or more atlases comprise a brain implant site atlas corresponding to the brain implant site of the patient.

30. A system comprising:
an implantable medical device comprising a lead, wherein the implantable medical device is implanted in a patient, and wherein the lead comprises a plurality of electrodes; and
a computing device for selecting one or more parameters for electrical stimulation therapy, the computing device comprising:
a memory to store data representative of an implant location of the lead in the patient;
a user interface; and
one or more processors configured to:
determine, based on the implant location, probabilities of effectiveness of electrical stimulation delivered via each of the electrodes,
present, via the user interface, a visual representation of the determined probabilities, and
receive, via the user interface, user input specifying one or more parameters to control delivery of electrical stimulation by an electrical stimulator to the patient.
